(19) United States
(12) Patent Application Publication
(10) Pub. No.: US 2024/0307177 A1
(43) Pub. Date: Sep. 19, 2024

(54) PROSTHETIC HEART VALVE

(71) Applicant: EDWARDS LIFESCIENCES CORPORATION, Irvine, CA (US)

(72) Inventor: Tamir S. Levi, Zikhron Yaakov (IL)

(21) Appl. No.: 18/677,406

(22) Filed: May 29, 2024

Related U.S. Application Data
(63) Continuation of application No. 18/048,393, filed on Oct. 20, 2022, now Pat. No. 12,023,242.
Application No. 18/048,393 is a continuation of application No. 16/674,357, filed on Nov. 5, 2019, now Pat. No. 11,484,406.
Application No. 16/674,357 is a continuation of application No. 15/808,599, filed on Nov. 9, 2017, now Pat. No. 10,463,484.
(60) Provisional application No. 62/423,599, filed on Nov. 17, 2016.

(51) Int. Cl. A61F 2/24; A61F 2/915

(52) U.S. Cl. CPC A61F 2/2418; A61F 2/2412; A61F 2/2409; A61F 2/2433; A61F 2/2436; A61F 2/2439; A61F 2/2454; A61F 2/2463; A61F 2/915; A61F 2002/91575; A61F 2210/0014; A61F 2220/0075; A61F 2230/0006; A61F 2250/0003; A61F 2250/0018

(57) ABSTRACT

Embodiments of a prosthetic heart valve are disclosed. An implantable prosthetic valve can include an annular frame having an inflow end, an outflow end and a central longitudinal axis extending from the inflow end to the outflow end. The valve includes an inner skirt, where a first portion of the inner skirt extends circumferentially around the central longitudinal axis along an inner surface of the frame and a second portion of the inner skirt extends circumferentially around the central longitudinal axis outside of the frame and axially beyond the inflow end of the frame such that an inflow end of the inner skirt is positioned upstream of and is axially spaced from the inlet end of the frame. The valve includes a valvular structure including two or more leaflets, where each of the two or more leaflets are secured to the inner skirt.

### CROSS REFERENCE TO RELATED APPLICATIONS

The present application is a continuation of U.S. patent application Ser. No. 18/048,393, filed Oct. 20, 2022, which is a continuation of U.S. patent application Ser. No. 16/674,357, filed Nov. 5, 2019, now U.S. Pat. No. 11,484,406, which is a continuation of U.S. patent application Ser. No. 15/808,599, filed Nov. 9, 2017, now U.S. Pat. No. 10,463,484, which claims the benefit of U.S. Provisional Patent Application No. 62/423,599, filed Nov. 17, 2016, all of which applications are incorporated by reference herein.

### FIELD

The present disclosure concerns embodiments of a prosthetic heart valve.

### BACKGROUND

The human heart can suffer from various valvular diseases. These valvular diseases can result in significant malfunctioning of the heart and ultimately require replacement of the native valve with an artificial valve. There are a number of known artificial valves and a number of known methods of implanting these artificial valves in humans.

Various surgical techniques may be used to replace or repair a diseased or damaged valve. Due to stenosis and other heart valve diseases, thousands of patients undergo surgery each year wherein the defective native heart valve is replaced by a prosthetic valve. Another less drastic method for treating defective valves is through repair or reconstruction, which is typically used on minimally calcified valves. The problem with surgical therapy is the significant risk it imposes on these chronically ill patients with high morbidity and mortality rates associated with surgical repair.

When the native valve is replaced, surgical implantation of the prosthetic valve typically requires an open-chest surgery during which the heart is stopped and patient placed on cardiopulmonary bypass (a so-called “heart-lung machine”). In one common surgical procedure, the diseased native valve leaflets are excised and a prosthetic valve is sutured to the surrounding tissue at the valve annulus. Because of the trauma associated with the procedure and the attendant duration of extracorporeal blood circulation, some patients do not survive the surgical procedure or die shortly thereafter. It is well known that the risk to the patient increases with the amount of time required on extracorporeal circulation. Due to these risks, a substantial number of patients with defective native valves are deemed inoperable because their condition is too frail to withstand the procedure. By some estimates, more than 50% of the subjects suffering from valve stenosis who are older than 80 years cannot be operated on for valve replacement.

Because of the drawbacks associated with conventional open-heart surgery, percutaneous and minimally-invasive surgical approaches have become widely adopted in recent years. In one technique, a prosthetic valve is configured to be implanted in a much less invasive procedure by way of catheterization. For instance, U.S. Pat. Nos. 5,411,522 and 6,730,118, which are incorporated herein by reference, describe collapsible transcatheter heart valves that can be percutaneously introduced in a compressed state on a catheter and expanded in the desired position by balloon inflation or by utilization of a self-expanding frame or stent.

An important design parameter of a transcatheter heart valve is the amount of mechanical interaction between the prosthetic frame or stent and the native anatomy at the annulus and left ventricle outflow tract (“LVOT”) level. It is desirable to reduce tissue trauma and/or mechanical stress in the native anatomy to avoid procedural-related injuries, such as LVOT, aortic and/or annulus rupture, which may occur in the region of the aortic root and the LVOT during transcatheter aortic valve replacement.

### SUMMARY

An exemplary embodiment of a prosthetic heart valve can include an annular frame having an inflow end, an outflow end and a central longitudinal axis extending from the inflow end to the outflow end. The frame can include a valvular structure including two or more leaflets, each of the two or more leaflets having a leaflet inflow edge positioned at least partially outside of the frame and a leaflet outflow edge positioned within the frame, wherein at least a portion of each of the leaflet inflow edges is unsupported by the frame. Some embodiments can include an inner skirt, wherein a first portion of the inner skirt extends circumferentially around the central longitudinal axis along an inner surface of the frame and a second portion of the inner skirt extends circumferentially around the central longitudinal axis outside of the frame. In some embodiments, the portions of the leaflet inflow edges unsupported by the frame are connected to the second portion of the inner skirt.

Additionally and/or alternatively, the inner skirt can include a first set of fibers that are sufficiently stiff to impede inward bending of the second portion due to systolic pressure gradient and blood flowing from the left ventricle to the aorta. In some embodiments, the first set of fibers run parallel to the central longitudinal axis. In some embodiments, the inner skirt includes a second set of fibers that run perpendicular to the first set of fibers, wherein the first set of fibers are stiffer than the second set of fibers. In some embodiments, the first set of fibers include monofilaments. In some embodiments, the portions of the leaflet inflow edges unsupported by the frame each include an apex portion of each leaflet. In some embodiments, the frame includes at least two rows of cells defining openings having a length in an axial direction and a length of the portion of the inflow edges unsupported by the frame is equal to or greater than the length of the openings. Additionally and/or alternatively, some embodiments can include an outer sealing member mounted on the outside of the frame.

Additionally and/or alternatively, some embodiments can include an outer support layer mounted on the outside of the frame having an inflow end portion that extends axially beyond the inflow end of the frame, and the portion of the leaflet inflow edges unsupported by the frame is connected to the inflow end portion of the outer support layer.

Some embodiments of an implantable prosthetic valve can include an annular frame having an inflow end, an outflow end and a central longitudinal axis extending from the inflow end to the outflow end, an inner skirt, wherein a first portion of the inner skirt extends circumferentially around the central longitudinal axis along an inner surface of the frame and a second portion of the inner skirt extends circumferentially around the central longitudinal axis outside of the frame and a valvular structure including two or more leaflets, each of the two or more leaflets having a leaflet inflow edge positioned at least partially outside of the frame and a leaflet outflow edge positioned within the frame, wherein at least a portion of each of the leaflet inflow edges are connected to the second portion of the inner skirt.

In some embodiments, the inner skirt includes a first set of fibers and a second set of fibers that runs perpendicular to the first set of fibers, wherein the first set of fibers is stiffer than the second set of fibers. In some embodiments, the frame includes a row of cells defining openings having a length in an axial direction and further wherein a length of the second portion is equal to or greater than the length of the openings. In some embodiments, an outer skirt can be connected to the second portion of the inner skirt. In some embodiments, the portions of the leaflet inflow edges unsupported by the frame include an apex portion of each leaflet.

Some embodiments of an implantable prosthetic valve can include an annular frame having an inflow end, an outflow end and a central longitudinal axis extending from the inflow end to the outflow end, a support layer, wherein a first portion of the support layer extends circumferentially around the central longitudinal axis along an outer surface of the frame and a second portion of the support layer extends circumferentially around the central longitudinal axis axially beyond the inflow end of the frame, and a valvular structure wherein at least a portion of the valvular structure is connected to the second portion of the support layer and is unsupported by the frame. In some embodiments, an outer skirt can be connected to the second portion of the support layer at an inflow end of the outer skirt. In some embodiments, the support layer includes a first set of fibers and a second set of fibers that runs perpendicular to the first set of fibers, wherein the first set of fibers is stiffer than the second set of fibers. In some embodiments, the valvular structure comprises a plurality of leaflets and the first set of fibers includes monofilaments. In some embodiments, the at least a portion of the valvular structure unsupported by the frame includes an apex portion of each leaflet.

The foregoing and other objects, features, and advantages of the disclosure will become more apparent from the following detailed description, which proceeds with reference to the accompanying figures.

### BRIEF DESCRIPTION OF THE DRAWINGS

FIG. 1 shows a front view of an exemplary embodiment of a prosthetic heart valve.

FIG. 2 shows a side view of the prosthetic heart valve of FIG. 1 with the outer skirt removed to show an inner skirt and valvular structure mounted on the frame.

FIG. 3 shows a rotated front view of the prosthetic heart valve of FIG. 2.

FIG. 4 shows an enlarged, partial cross-sectional view of a prosthetic heart valve, according to one embodiment.

FIG. 5A shows an enlarged, partial cross-sectional view of a prosthetic heart valve, according to one embodiment.

FIG. 5B shows a side view of an exemplary support layer shown in flattened configuration.

FIG. 6 shows a top plan view of the prosthetic heart valve of FIG. 1.

FIG. 7 shows a side view of an exemplary inner skirt shown in flattened configuration.

FIG. 8 shows a side view of an exemplary leaflet shown in flattened configuration.

FIG. 9 shows a side view of an exemplary outer skirt shown in flattened configuration.

FIG. 10 shows a perspective view of an exemplary frame of the prosthetic heart valve of FIG. 1.

FIG. 11 shows a side view of the frame of FIG. 10 shown in flattened configuration.

FIG. 12 is a side view of the prosthetic valve of FIG. 1 in a collapsed configuration mounted on the balloon of a delivery apparatus.

### DETAILED DESCRIPTION

For purposes of this description, certain aspects, advantages, and novel features of the embodiments of this disclosure are described herein. Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any particular embodiments. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

As used herein, the term “coupled” generally means physically coupled or linked and does not exclude the presence of intermediate elements between the coupled items absent specific contrary language.

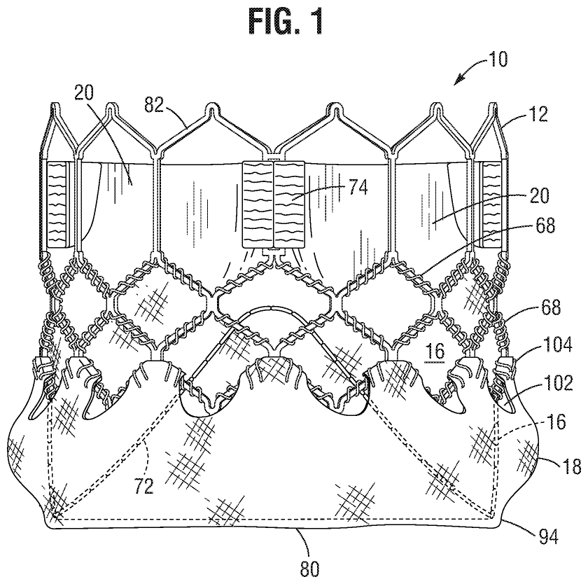

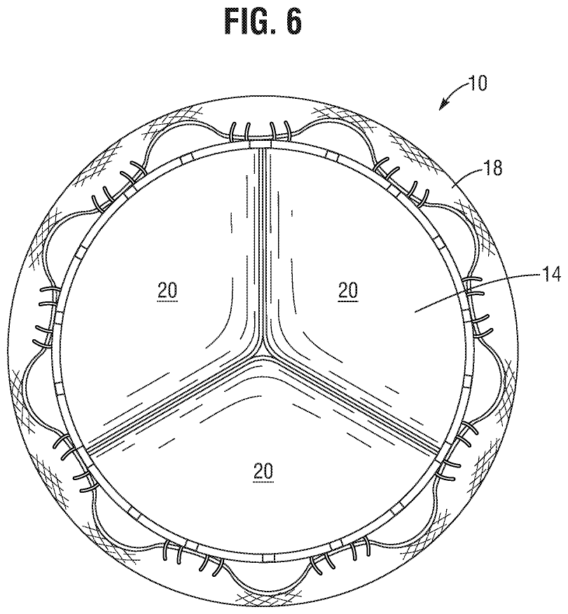

FIGS. 1 and 6 show side and top plan views, respectively, of a prosthetic heart valve 10 having an inflow end 80 and an outflow end 82, according to one embodiment. The illustrated prosthetic valve is adapted to be implanted in the native aortic annulus, although in other embodiments it can be adapted to be implanted in the other native annuluses of the heart (the mitral valve, pulmonary valve and triscupid valve). The prosthetic valve 10 can have one or more of the following components: a stent, or frame, 12, a valvular structure 14 and an inner skirt, or sealing member, 16. The valve 10 can also include an outer skirt, or sealing member 18.

The valvular structure 14 can comprise three leaflets 20, collectively forming a valvular structure, which can be arranged to collapse in a tricuspid arrangement, as best shown in FIG. 6.

The lower edge of valvular structure 14 desirably has an undulating, curved scalloped shape. By forming the leaflets with this scalloped geometry, stresses on the leaflets are reduced, which in turn improves durability of the valve. Moreover, by virtue of the scalloped shape, folds and ripples at the belly of each leaflet (the central region of each leaflet), which can cause early calcification in those areas, can be eliminated or at least minimized. The scalloped geometry also reduces the amount of tissue material used to form the valvular structure 14, thereby allowing a smaller, more even crimped profile at the inflow end of the valve. The leaflets 20 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

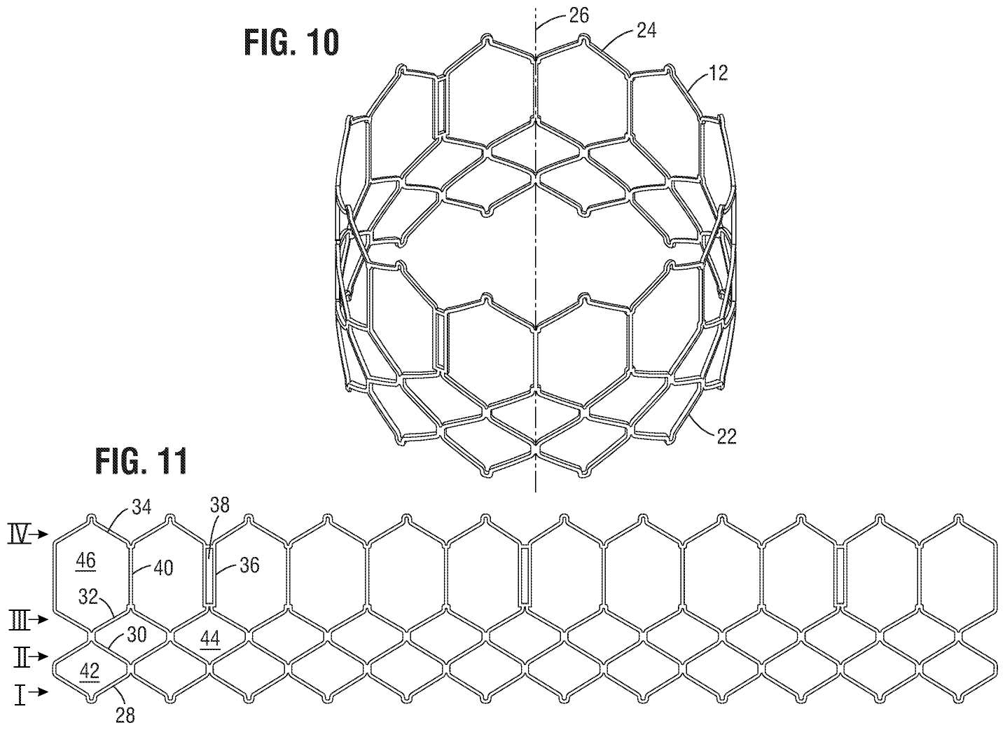

The bare frame 12 is shown in FIGS. 10 and 11. The frame 12 has an inflow end 22, an outflow end 24 and a central longitudinal axis 26 extending from the inflow end 22 to the outflow end 24. The frame 12 can be made of any of various suitable plastically-expandable materials (e.g., stainless steel, etc.) or self-expanding materials (e.g., Nitinol) as known in the art.

When constructed of a plastically-expandable material, the frame 12 (and thus the prosthetic valve 10) can be crimped to a radially compressed state on a delivery catheter and then expanded inside a patient by an inflatable balloon or equivalent expansion mechanism. When constructed of a self-expandable material, the frame 12 (and thus the prosthetic valve 10) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frame 12 include, without limitation, stainless steel, a nickel based alloy (e.g., a cobalt-chromium or a nickel-cobalt-chromium alloy), polymers, or combinations thereof. In particular embodiments, frame 12 is made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight. It has been found that the use of MP35N to form frame 12 provides superior structural results over stainless steel. In particular, when MP35N is used as the frame material, less material is needed to achieve the same or better performance in radial and crush force resistance, fatigue resistances, and corrosion resistance. Moreover, since less material is required, the crimped profile of the frame can be reduced, thereby providing a lower profile valve assembly for percutaneous delivery to the treatment location in the body.

Referring to FIGS. 10 and 11, the frame 12 in the illustrated embodiment comprises a first, lower row I of angled struts 28 arranged end-to-end and extending circumferentially at the inflow end of the frame; a second row II of circumferentially extending, angled struts 30; a third row III of circumferentially extending, angled struts 32; and a forth row IV of circumferentially extending, angled struts 34 at the outflow end of the frame 12. The forth row IV of angled struts 34 can be connected to the third row III of angled struts 32 by a plurality of axially extending window frame portions 36 (which define commissure windows 38) and a plurality of axially extending struts 40. Each axial strut 40 and each frame portion 36 extends from a location defined by the convergence of the lower ends of two angled struts 34 to another location defined by the convergence of the upper ends of two angled struts 32.

Each commissure window frame portion 36 mounts a respective commissure 74 of the leaflet structure 14. As can be seen, each frame portion 36 is secured at its upper and lower ends to the adjacent rows of struts to provide a robust configuration that enhances fatigue resistance under cyclic loading of the valve compared to known cantilevered struts for supporting the commissures of the leaflet structure. This configuration enables a reduction in the frame wall thickness to achieve a smaller crimped diameter of the valve. In particular embodiments, the thickness of the frame 12 measured between the inner diameter and outer diameter is about 0.48 mm or less.

The struts and frame portions of the frame collectively define a plurality of open cells of the frame. At the inflow end 22 of the frame 12, struts 28 and struts 30 define a lower row of cells defining openings 42. The second and third rows of struts 30 and 32, respectively, define an intermediate row of cells defining openings 44. The third and fourth rows of struts 32 and 34, along with frame portions 36 and struts 40, define an upper row of cells defining openings 46. The openings 46 are relatively large and are sized to allow portions of the valvular structure 14 to protrude, or bulge, into and/or through the openings 46 when the frame 12 is crimped in order to minimize the crimping profile.

The frame 12 can have other configurations or shapes in other embodiments. For example, the frame 12 can comprise a plurality of circumferential rows of angled struts 28, 30, 32, 34 connected directly to each other without vertical struts 40 or frame portions 36 between adjacent rows of struts, or the rows of struts 28, 30, 32, 34 can be evenly spaced with vertical struts 40 and/or frame portions 36 extending therebetween. In other embodiments, the frame can comprise a braided structure braided from one or more metal wires.

The inner skirt 16 can have a plurality of functions, which can include to assist in securing the valvular structure 14 and/or the outer skirt to the frame 12 and to assist in forming a good seal between the valve 10 and the native annulus by blocking the flow of blood below the lower edges of the leaflets. The inner skirt 16 can comprise a tough, tear resistant material such as polyethylene terephthalate (PET), although various other synthetic or natural materials can be used. The thickness of the skirt desirably is less than 6 mil or 0.15 mm, and desirably less than 4 mil or 0.10 mm, and even more desirably about 2 mil or 0.05 mm. In particular embodiments, the skirt 16 can have a variable thickness, for example, the skirt can be thicker at its edges than at its center. In one implementation, the skirt 16 can comprise a PET skirt having a thickness of about 0.07 mm at its edges and about 0.06 mm at its center. The thinner skirt can provide for better crimping performances while still providing good perivalvular sealing.

As noted above, the valvular structure 14 in the illustrated embodiment includes three flexible leaflets 20 (although a greater or fewer number of leaflets can be used). An exemplary leaflet 20 is shown in FIG. 8. Each leaflet 20 can have a reinforcing strip 58 secured (e.g., sewn) to the inner surface of the lower edge portion 56. Each leaflet 20 in the illustrated configuration can have an upper (outflow) free edge 48 extending between opposing upper tabs 50 on opposite sides of the leaflet 20. Below each upper tab 50 there can be a notch 52 separating the upper tab 50 from a corresponding lower tab 54. The lower (inflow) edge portion 56 of the leaflet extending between respective ends of the lower tabs 54 can include vertical, or axial, edge portions 60 on opposites of the leaflets extending downwardly from corresponding lower tabs 54 and a substantially V-shaped, intermediate edge portion 62 having a smooth, curved apex portion 64 at the lower end of the leaflet and a pair of oblique portions 66 that extend between the axial edge portions 60 and the apex portion 64. The oblique portions 66 can have a greater radius of curvature than the apex portion 64. The leaflets can have various other shapes and/or configurations in other embodiments. For example, the leaflets need not have a V-shaped or scalloped inflow edges and instead each leaflet can have a square or rectangular shape defining a straight inflow edge.

The leaflets 20 can be sutured together to form the assembled valvular structure 14, which can then be secured to the frame 12. For example, the leaflets 20 can be secured to one another at their adjacent sides to form commissures 74 of the valvular structure. A plurality of flexible connectors (not shown) can be used to interconnect pairs of adjacent sides of the leaflets and to mount the leaflets to the commissure window frame portions 30, as further discussed below. The leaflets 20 can additionally and/or alternatively be secured together via adjacent sub-commissure portions (not shown) of two leaflets that can be sutured directly to each other.

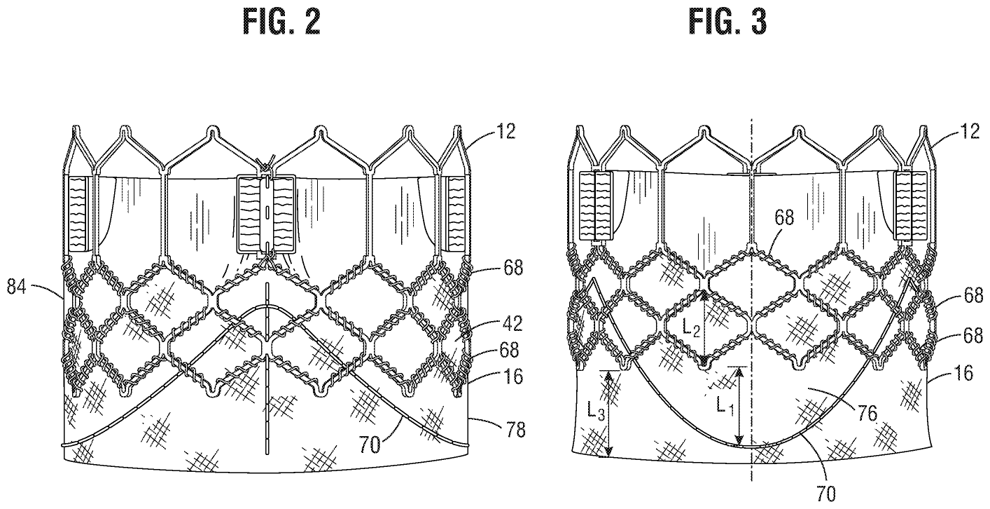

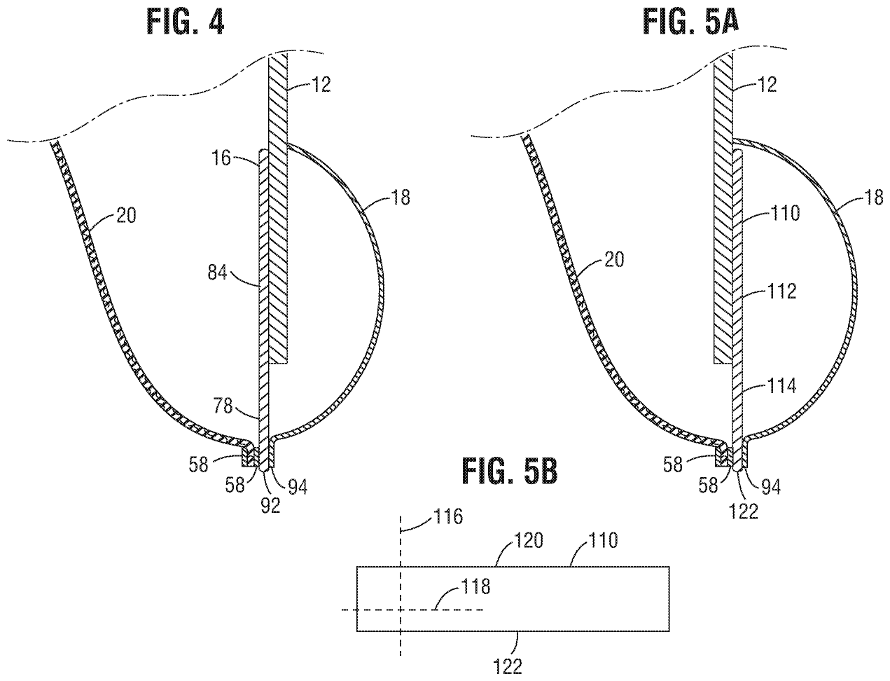

FIGS. 2 and 3 show the frame 12, the valvular structure 14 and the inner skirt 16 after securing the valvular structure 14 to the inner skirt 16 and then securing these components to the frame 12. The inner skirt 16 can be secured to the inside of frame 12 via sutures 68. The valvular structure 14 can be attached to the inner skirt 16 via the one or more thin PET reinforcing strips 58 along the lower (inflow) edge portions 56 of the leaflets 20. The reinforcing strips 58 collectively can form a sleeve, which can enable a secure suturing and protect the pericardial tissue of the valvular structure 14 from tears. Valvular structure 14 can be sandwiched between the inner skirt 16 and the thin PET strips 58. Sutures 70, which secure the PET strips and the valvular structure 14 to inner skirt 16, can be any suitable suture, such as an Ethibond suture. Sutures 70 desirably track the curvature of the bottom edge of valvular structure 14 and are collectible referred to as scallop line 72. In lieu of or in addition to the strips 58 along the inner surface of the leaflets, reinforcing strips 58 can be positioned between the inner skirt 16 and the inflow edge portions 56 of the leaflets (FIG. 4).

As shown in FIGS. 2 and 3, portions of the inflow edges 56 of the leaflets 56 are positioned between diagonal lines defined by the struts 28, 30 of the first and second circumferential rows of the struts. In some embodiments, the inflow edges 56 of the leaflets 20 can track the diagonal lines defined by the struts 28, 30. In such embodiments, the portions of the inflow edges 56 above the inflow end of the frame can be sutured to adjacent struts 28, 30.

The outflow end portion of the valvular structure 14 can be secured to the window frame portions 36. In particular, each leaflet 20 can have opposing tab portions, each of which is paired with an adjacent tab portion of another leaflet to form a commissure 74. As best shown in FIG. 1, the commissures 74 can extend through windows 38 of respective window frame portions 36 and sutured in place. Further details of the commissures 74 and a method for assembling the commissures and mounting them to the frame are disclosed in U.S. Publication No. 2012/0123529, which is incorporated herein by reference. The inner skirt 16 can terminate short of the window frame portions 36 and does not extend the entire length of the frame 12. In alternative embodiments, the inner skirt 16 can extend the entire length or substantially the entire length of the frame 12 from below the inflow end 22 to the outflow end 24. In other embodiments, the inner skirt 16 can extend up to the second row of angled struts 30.

In particular embodiments, and as shown in FIGS. 1-3, the valvular structure 14 can extend axially beyond the inflow end 22 of the frame 12 such that at least a portion of the inflow edges 56 of each leaflet is not supported by the frame 12. For example, the apex portions 64 of the leaflets 20 can extend axially beyond and can be unsupported by the frame 12. Additionally and/or alternatively, the oblique portions 66 and the apex portions 64 can extend axially beyond and can be unsupported by the frame 12. Additionally and/or alternatively, the intermediate portions 62, the oblique portions 66 and the apex portions 64 can extend axially beyond and can be unsupported by the frame 12. Additionally and/or alternatively, the vertical edge portions 60, the intermediate portions 62, the oblique portions 66 and the apex portions 64 can extend axially beyond and can be unsupported by the frame 12. In some embodiments, a portion 76 of each leaflet extends below the inflow end 22 of the frame a length L₁ that is equal to or greater than a length L₂ of each cell opening 42 in the axial direction. In some embodiments, L₁ can range between about 2 mm to 8 mm in length, with 4 mm being a specific example. Additionally and/or alternatively, L₁ can be up to 12 mm. In some embodiments, L₂ can range between about 3 mm to 5 mm in length, with 4 mm being a specific example. Additionally and/or alternatively, L₂ can range between 2 mm to 7 mm.

Additionally and/or alternatively, as shown in FIG. 4, the inner skirt 16 can include a first portion 84, also referred to as supported portion, that extends circumferentially around the central longitudinal axis of the prosthetic valve along an inner surface of the frame 12 and a second portion, also referred to as unsupported portion 78, at the inflow end 80 of the prosthetic valve 10. The unsupported portion 78 is unsupported by the frame 12. The unsupported portion 78 can extend circumferentially around the central longitudinal axis of the prosthetic valve to form an unsupported circumference at the inflow end 80. As shown in FIG. 4, the portion of each leaflet 20 that is not supported by the frame 12 can be connected to the inner skirt 16. In some embodiments, a length L₃ of the unsupported portion 78 is equal to or greater than the length L₂ of the openings 42. In some embodiments, L₃ can be 2 mm to 3 mm longer than L₁. Additionally and/or alternatively, L₃ can range from the same length as L₁ to 5 mm longer than L₁. In some embodiments, L₃ can range between about 2 mm to 11 mm in length, with 6 mm being a specific example. Additionally and/or alternatively, L₃ can be up to 15 mm.

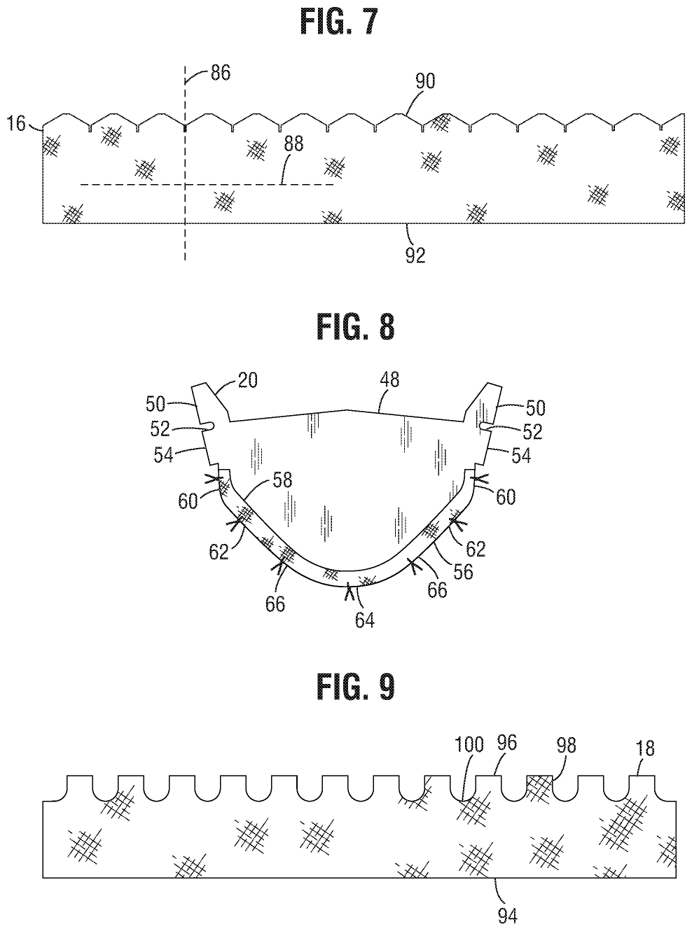

In some embodiments, as best shown in FIG. 7, the inner skirt 16 can be woven from a first set of fibers 86, or yarns or strands, and a second set of fibers 88, or yarns or strands. The first and set of fibers 86, 88 can run perpendicular and parallel, respectively, to upper 90 and lower 92 edges of the inner skirt 16, or alternatively, they can extend at angles between 0 and 90 degrees relative to the upper and lower edges 90, 92 of the inner skirt 16. The first set of fibers 86 can be stiffer than the second set of fibers 88. For example the first set of fibers 86 can include monofilaments. The extra stiffness of the first set of fibers 86 can provide support to the prosthetic leaflets 20 during systole. For example, the first set of fibers 86 can reinforce the inner skirt 16 to impede inward and/or outward folding or bending of the unsupported portion 78 of the inner skirt and/or the unsupported portions of 76 of the leaflets that are unsupported by the frame 12 due to systolic pressure gradient and blood flowing from the left ventricle to the aorta. In other embodiments, the fibers 86 can be thicker than the fibers 88 and/or axially extending reinforcing wires (extending in the same direction as fibers 86) can be woven or secured to the skirt to increase the axial stiffness of the skirt. The reinforcing wires can be metal wires formed from a suitable biocompatible metal, such as stainless steel or Nitinol.

In some embodiments, the first set of fibers 86 and the second set of fibers 88 can extend at angles of about 45 degrees relative to the upper and lower edges. The inner skirt 16 can be formed by weaving the fibers at 45 degree angles relative to the upper and lower edges of the fabric. Alternatively, the skirt can be diagonally cut from a vertically woven fabric (where the fibers extend perpendicular to the edges of the material) such that the fibers extend at 45 degree angles relative to the cut upper and lower edges of the skirt. The opposing short edges of the inner skirt can be non-perpendicular to the upper and lower edges.

FIG. 9 shows a flattened view of the outer skirt 18 prior to its attachment to the inner skirt 16 and the frame 12. The outer skirt 18 can be laser cut or otherwise formed from a strong, durable piece of material, such as woven PET, although other synthetic or natural materials can be used. The outer skirt 18 can have a substantially straight lower edge 94 and an upper edge 96 defining a plurality of alternating projections 98 and notches 100. Alternatively, the lower edge 94 of the outer skirt 18 can include openings similar to those on the upper edge, or can include a separate row of holes adjacent to the straight lower edge 94. As best shown in FIG. 1, the lower edge 94 of the outer skirt 18 can be secured to the lower edge 92 of the inner skirt 16 at the inflow end of the prosthetic valve, such as by sutures, welding, and/or an adhesive. In particular embodiments, the lower edge 94 of the outer skirt 18 is tightly sutured or otherwise secured (e.g., by welding or an adhesive) to the inner skirt 16.

The upper edge 96 of the outer skirt 18 desirably is secured to the frame 12 and/or the inner skirt 16 at spaced-apart locations around the circumference of the frame 12. In the illustrated embodiment, for example, the projections 98 of the outer skirt can be sutured to the struts of the frame 12 and/or the inner skirt 16. As shown, the corners of the projections 98 of the outer skirt 18 can be folded over respective struts and secured with sutures 104 (FIG. 1). The notches 100 can remain unattached to the inner skirt 16 and the frame 12. When the valve 10 is deployed within the body (e.g., within the native aortic valve), the outer skirt 18 can cooperate with the inner skirt 16 to prevent or at least minimize paravalvular leakage.

The absence of metal components of the frame or other rigid members along the inflow end portion of the prosthetic valve advantageously reduces mechanical compression of the native valve annulus (e.g., the aortic annulus) and the left ventricular outflow tract (when implanted in the aortic position), thus reducing the risk of trauma to the surrounding tissue.

As shown in FIG. 5A, some embodiments of a prosthetic valve 10 do not include an inner skirt 16 and instead can include a support layer 110 disposed radially between the frame 12 and the outer skirt 18. In this manner, the support layer 110 and the outer skirt 18 form an outer sealing member with the support layer 110 forming an inner wall or layer of the sealing member and the outer skirt forming an outer wall or layer of the sealing member. A first portion 112 of the support layer 110 can extend circumferentially around the central longitudinal axis of the prosthetic valve along an outer surface of the frame 12 and a second portion 114 of the support layer 110 can extend circumferentially around the central longitudinal axis axially beyond the inflow end 22 of the frame. The unsupported portions 76 of the leaflets can be connected to the support layer 110 (e.g., by sutures, an adhesive, and/or welding) similar to the manner that the leaflets are connected to the inner skirt in the embodiment of FIG. 4. For example, the inflow edge portions 56 can be sutured to reinforcing strips 58 and the support layer 110 with the inflow edge portions 56 sandwiched between the support layer 110 and the reinforcing strips 58. Additionally and/or alternatively, reinforcing strips 58 can be positioned between the inflow edge portions 56 and the support layer 110 with sutures securing these layers together. Additionally and/or alternatively, the outer skirt 18 can be connected to the second portion 114 of the support layer (e.g., by sutures, an adhesive, and/or welding).

The support layer 110 can have the same or similar size and shape as the inner skirt 16 and can be made of the same or similar materials as the inner skirt 16. For example, as shown in FIG. 5B, the support layer 110 can be woven from a first set of fibers 116 and a second set of fibers 118, or yarns or strands. The first and second set of fibers 116, 118 can run perpendicular and parallel, respectively, to upper and lower edges 120, 122 of the support layer 110, or alternatively, they can extend at angles between 0 and 90 degrees relative to the upper and lower edges 120, 122 of the support layer. The first set of fibers 116 can be stiffer than the second set of fibers 118. For example the first set of fibers 116 can include monofilaments, reinforcing wires and/or thicker fibers than the fibers 118. The extra stiffness of the first set of fibers 116 can provide support to the prosthetic leaflets 20 during systole. For example, the first set of fibers 116 can reinforce the support layer 110 to impede inward and/or outward folding or bending of the lower portion 114 and the unsupported portions 76 of the leaflets due to systolic pressure gradient and blood flowing from the left ventricle to the aorta.

In alternative embodiments, the prosthetic valve 10 can include an inner skirt 16 and an outer support layer 110.

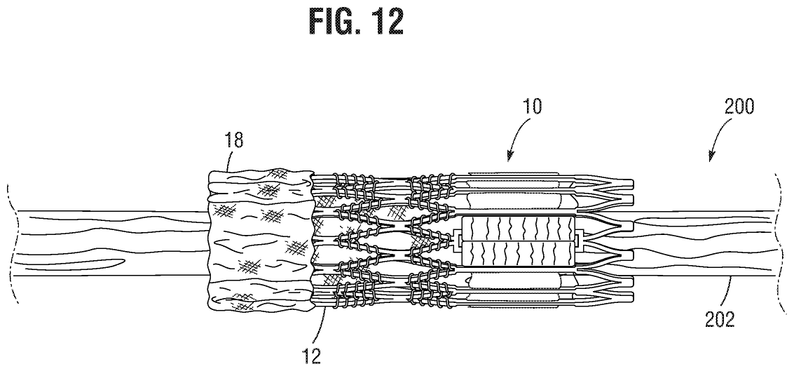

FIG. 12 shows the prosthetic valve 10 in a radially compressed state for delivery into a patient's body on a delivery catheter 200. As shown, the prosthetic valve 10 can be crimped on a balloon 202 of the delivery catheter 200. In the delivery configuration, the outer skirt 18 can be folded against the outer surface of the frame 12. When deployed inside the body (e.g., after being released from the sheath of the delivery catheter), the stent 12 and the outer skirt 18 can radially expand, such as by inflating the balloon 202.

In view of the many possible embodiments to which the principles of the disclosed technology may be applied, it should be recognized that the illustrated embodiments are only preferred examples of the disclosure and should not be taken as limiting the scope of the disclosure. Rather, the scope of the disclosure is defined by the following claims. I therefore claim as my disclosure all that comes within the scope and spirit of these claims.

We claim:

1. An implantable prosthetic valve for replacing a native valve of the heart, the prosthetic valve comprising:
an annular frame having an inflow end, an outflow end and a central longitudinal axis extending from the inflow end to the outflow end, wherein the frame is expandable from a radially compressed state to a radially expanded state in which the prosthetic valve engages an annulus of the native valve;
an inner skirt, wherein a first portion of the inner skirt extends circumferentially around the central longitudinal axis along an inner surface of the frame and a second portion of the inner skirt extends circumferentially around the central longitudinal axis outside of the frame, and wherein the second portion of the inner extends axially beyond the inflow end of the frame such that an inflow end of the inner skirt is positioned upstream of and is axially spaced from the inlet end of the frame; and
a valvular structure including two or more leaflets, wherein each of the two or more leaflets is secured to the inner skirt.

2. The prosthetic valve of claim 1, wherein each of the two or more leaflets is secured to the first portion of the inner skirt with sutures.

3. The prosthetic valve of claim 1, further comprising an outer skirt positioned radially outside of the frame and connected to the second portion of the inner skirt at an inflow end of the outer skirt.

4. The prosthetic valve of claim 3, wherein the inflow end of the outer skirt and the inflow end of the inner skirt are connected by stitches that are positioned upstream of the inlet end of the frame.

5. The prosthetic valve of claim 3, wherein the outer skirt extends radially outward from the frame when the frame is in the radially expanded state.

6. The prosthetic valve of claim 1, wherein the frame comprises a plurality of interconnected angled struts arranged into circumferentially extending rows of angled struts.

7. The prosthetic valve of claim 6, wherein the second portion of the inner skirt extends axially away from at least a portion of the plurality of interconnected angled struts.

8. The prosthetic valve of claim 6, wherein the first portion of the inner skirt is disposed against inner surfaces of a portion of angled struts of the plurality of interconnected angled struts.

9. The prosthetic valve of claim 1, further comprising reinforcing strips secured to inflow edge portions of the two or more leaflets.

10. The prosthetic valve of claim 9, wherein the reinforcing strips comprises PET.

11. The prosthetic valve of claim 9, wherein the reinforcing strips are attached to the inner skirt.

12. The prosthetic valve of claim 9, wherein the inflow edge portions of the two or more leaflets are positioned radially between the reinforcing strips and the inner skirt.

13. The prosthetic valve of claim 1, wherein the inner skirt includes a first set of fibers and a second set of fibers that runs perpendicular to the first set of fibers, and wherein the first set of fibers is stiffer than the second set of fibers.

14. An implantable prosthetic valve for replacing a native valve of the heart, the prosthetic valve comprising:
an annular frame having an inflow end, an outflow end and a central longitudinal axis extending from the inflow end to the outflow end, wherein the frame is expandable from a radially compressed state to a radially expanded state in which the prosthetic valve engages an annulus of the native valve;
an inner skirt, wherein a first portion of the inner skirt extends circumferentially around the central longitudinal axis along an inner surface of the frame and a second portion of the inner skirt extends circumferentially around the central longitudinal axis outside of the frame, wherein the first portion of the inner skirt is connected to the frame with sutures, and the second portion of the inner skirt is not sutured to the frame;
a valvular structure including two or more leaflets, wherein each of the two or more leaflets are sutured to the first portion of the inner skirt; and
an outer skirt extending circumferentially around the central longitudinal axis along an outer surface of the frame, wherein the outer skirt is secured to the second portion of the inner skirt with stitches that are positioned upstream of and axially spaced from the inflow end of the frame.

15. The prosthetic valve of claim 14, wherein the frame comprises a plurality of interconnected angled struts arranged into circumferentially extending rows of angled struts, and wherein the first portion of the inner skirt is disposed against inner surfaces of the portion of angled struts of the plurality of interconnected angled struts, and the second portion of the inner skirt extends outside of the frame and is unsupported by the frame.

16. The prosthetic valve of claim 14, wherein the inner skirt includes a first set of fibers and a second set of fibers that runs perpendicular to the first set of fibers, and wherein the first set of fibers is stiffer than the second set of fibers.

17. The prosthetic valve of claim 14, further comprising reinforcing strips arranged along inflow edges of the two or more leaflets, wherein the reinforcing strips are positioned radially inside of the leaflets.

18. An implantable prosthetic valve for replacing a native valve of the heart, the prosthetic valve comprising:
an annular frame having an inflow end, an outflow end and a central longitudinal axis extending from the inflow end to the outflow end, wherein the frame is expandable from a radially compressed state to a radially expanded state;
a first layer of fabric, wherein a first portion of the first layer of fabric extends circumferentially around the central longitudinal axis along an inner surface of the frame;
a second layer of fabric; and
a valvular structure including two or more leaflets, wherein each of the two or more leaflets are arranged between the first layer of fabric and the second layer of fabric, and wherein portions of the first and second layers of fabric extend outside of the frame and axially beyond the inflow end of the frame such that the portions of the first and second fabric layers are upstream of the inflow end of the frame.

19. The prosthetic valve of claim 18, further comprising an outer skirt positioned radially outside of the frame, wherein an inflow end portion of the outer skirt extends axially beyond the inflow end of the frame and is connected to the portions of the first and second layers of fabric that are outside the frame.

20. The prosthetic valve of claim 19, wherein the outer skirt extends radially outward from the frame, relative to the central longitudinal axis, when the frame is in the radially expanded state.

21. The prosthetic valve of claim 20, wherein the two or more leaflets are sutured to the first and second layers of fabric.
